(19) United States
(12) Patent Application Publication
(10) Pub. No.: US 2013/0125309 A1
(43) Pub. Date: May 23, 2013

(54) PATIENT TABLE AND METHOD FOR THE PRODUCTION THEREOF

(71) Applicants: Peter Schmuhl, Liebschuetz (DE); Michael Weinholdt, Jena (DE); Uwe Weinzierl, Jena (DE); J.A.D. Backer, St. Oedenrode (NL)

(72) Inventors: Peter Schmuhl, Liebschuetz (DE); Michael Weinholdt, Jena (DE); Uwe Weinzierl, Jena (DE); J.A.D. Backer, St. Oedenrode (NL)

(73) Assignee: SCHMUHL FASERVERBUNDTECHNIK GMBH & CO. KG, Liebschuetz (DE)

(21) Appl. No.: 13/504,514

(22) PCT Filed: Oct. 21, 2010
(86) PCT No.: PCT/DE10/50079

§ 371 Date: Aug. 14, 2012

(30) Foreign Application Priority Data
Oct. 27, 2009 (DE) 10 2009 051 048.6

(51) Int. Cl. A61B 6/04

(52) U.S. Cl. CPC A61B 6/0442
USPC 5/601; 156/245

(57) ABSTRACT

A patient table for diagnostic equipment includes at least one self-reinforced plastic.

### CROSS-REFERENCE TO PRIOR APPLICATIONS

This application is a U.S. National Phase application under 35 U.S.C. §371 of International Application No. PCT/DE2010/050079, filed on Oct. 21, 2010, and claims benefit to German Patent Application No. DE 10 2009 051 048.6, filed on Oct. 27, 2009. The International Application was published in German on May 5, 2011 as WO 2011/050794 under PCT Article 21(2).

### FIELD

The present invention is directed to a patient table for diagnostic and medical treatment equipment.

### BACKGROUND

In medical technology, patient tables are used in a variety of ways in connection with diagnostic devices or treatment equipment. Particularly in connection with imaging examinations, e.g., magnetic resonance spectroscopy (MR) or computer tomography, high requirements are set for the patient table with respect to strength and stiffness. This is necessary so that the patients can be stably supported in one position during the examination period, which can last from several minutes to an hour, so as not to allow the results of the examination to be rendered inaccurate or even unusable. The patient tables must also be strong enough not to break under loads exerted by the patient. Patient tables of this kind must possess high strength and stiffness while maintaining small wall thicknesses because the available space for installing the patient tables in diagnostic equipment is often very limited and the table should weigh as little as possible. Patient tables made of thermoplastics such as those described in DE 44 16 202 A1 do not meet the requirements for high stiffness because of the low modulus of elasticity of a maximum 4 GPa of thermoplastics.

Further, the patient tables should not negatively influence the results of the medical examination. Negative influences on examination results can occur in MR, e.g., when the patient table contains metallically or electrically conductive materials and in CT when the table is made of a material which is highly absorbent for X-rays.

Therefore, for reasons of mechanical strength and stiffness, patient tables for diagnostic equipment are often produced from fiber reinforced composite materials.

Patient tables may include fiber reinforced composite materials. For example, a table of glass fiber reinforced plastic (GFRP) is described in DE 38 33 594 A1. The table comprises a bidirectionally formed material having a modulus of elasticity of approximately 15 GPa or a unidirectionally formed material with a modulus of elasticity of 30 GPa. A table constructed from these materials satisfies the requirement for high strength and stiffness. However, it is very heavy because the material has a density of about 2,000 kg/gm³. It is used in MR equipment because GFRP is not an electrically conductive material. It is not used in CT equipment. The reason for this is that glass fibers are highly absorbent for X-radiation.

Other patient tables are produced from carbon fiber reinforced plastic (CFRP). A table of this kind and the use thereof is described in EP 0 017 454 A1. Tables of this kind are very lightweight and have high strength and stiffness. At the same time, the absorption of X-rays is very low. However, the table is electrically conductive due to the use of carbon fibers. When this table is used in MR equipment, the electrical conductivity leads to field distortions in the main magnetic field and, therefore, to impairment of the results of the patient examination.

DE 197 31 234 A1 describes a patient support device comprising a fiber reinforced composite material which is produced using fibers of aromatic polyamides. However, the aromatic polyamides are a very expansive fiber material and have less stiffness and compressive strength compared to carbon fibers and glass fibers. As a result, patient tables produced therefrom with the very small wall thicknesses which are often necessary for application in diagnostic equipment do not possess the necessary stiffness and are also very expensive. Further, polyamides absorb moisture from the atmosphere which in turn has a negative effect on the electrical properties of the material of the patent support device.

A large number of patient tables are produced from fiber reinforced composite materials. A fiber reinforced composite material is a multiphase material or mixed material generally comprising two main components (an embedding matrix and reinforcing fibers). The reinforcing fibers and the embedding matrix assume very specific functions. Through mutual interaction of the two components, the fiber reinforced composite material acquires characteristics of a higher order than either of the two individual constituent components. However, this also causes the two constituent materials to have very different properties. Thus, the fibers can be electrically conductive or insulating. On the other hand, the fibers have a different density than the matrix materials. As a result of these very different properties, patient tables are either suited only for a specific diagnostic examination method or the diagnostic findings are negatively affected by the heterogeneous construction of the fiber reinforced composite materials.

Accordingly, all patient tables of this kind have the disadvantage that they are not equally suited to all common medical examination methods, particularly MR and CT.

### SUMMARY

In an embodiment, the present invention provides a patient table for diagnostic equipment including at least one self-reinforced plastic.

### DETAILED DESCRIPTION

In an embodiment, the present invention provides a possibility for supporting patients stably in one position for all common medical diagnostic methods equally during the examination period.

According to an embodiment of the invention, self-reinforced plastics, particularly self-reinforced polyolefins and most especially self-reinforced polyethylene are used as material for producing a patient table. Ribbon-shaped, highly stretched monofilaments in particular have an array of properties which have very advantageous results for the production of patient tables for diagnostic equipment.

They are characterized in that they have a modulus of elasticity of greater than 30 GPa, in some cases up to 70 GPa or higher, at a density of <1000 kg/m³ depending on the degree of stretching and on the polyethylene that is used. The degree of stretching can be 15:1 to 60:1. At the same time, they are not electrically conductive and exhibit very low absorption of X-rays and an extremely low moisture absorption.

For producing structural components from these materials, the ribbon-shaped, highly stretched monofilaments can be joined to type-specific plastics. This joining process can correspond to the principle of hot-melt adhesion or sintering. The proportion of ribbon-shaped, highly stretched monofilaments in composite bodies of this type is appreciably greater than 95%. The composite bodies produced in this way can be adapted to specific requirements of the respective equipment by deliberate orientation of the ribbon-shaped monofilaments in the structural component.

Accordingly, in a specific embodiment form, the monofilaments can be arranged in equal proportions in the 0° (longitudinal) direction, 90° (transverse) direction and ±45° direction of the patient table. The table then has the same characteristics in all directions and can therefore withstand longitudinal loads, transverse loads and torsional loads equally well.

However, it is also possible to arrange the monofilaments exclusively in 0° direction. A patient table produced in this way is characterized by a particularly high strength and stiffness in longitudinal direction.

Further, it is possible to use the monofilaments as cover layer for a sandwich construction. Any commercially available hard foam material can be used as core for a sandwich construction of this type. A patient table produced in this way according to the invention is characterized by extremely low weight.

The invention will be described more fully in the following with reference to an embodiment example. Kaypla® 525H Unidirectional HDPE (Teijin Monofilament Germany GmbH, Germany) having a basis weight of 105 g/m² was used to produce a patient table according to the invention. The material has a modulus of elasticity of 50 GPa and a density of <950 kg/m³. To construct the patient table, individual layers of this material were draped in layers on a female mold. The layer construction in this embodiment comprises a symmetrical construction, starting from the outside, of 1 layer in longitudinal direction (0° direction), 12 layers alternately in +45° direction and −45° direction, 4 layers in 90° direction, and another 40 layers again in 0° direction up to the center of the structural component. The layer construction is now repeated from the inside out in reverse sequence, i.e., 40 layers in 0° direction, 4 layers in 90° direction, 12 layers alternately in +45° direction and −45° direction and, finally, 1 layer in 0° direction. Accordingly, the structural component in its entirety comprises 114 individual layers. Subsequently, the layer assembly constructed in this way is packed in a foil bag as typically used for vacuum construction of a glass fiber prepeg or carbon fiber prepeg for autoclave curing. The entire assembly is evacuated at a pressure of up to 2 mbar, inserted into an autoclave and thermoformed at an autoclave pressure of 9.5 bar and a temperature of 130° C. After two hours, the layer assembly is cooled under pressure to below 65° C. Subsequently, the structural component part can be ejected from the mold. The finished structural component part has a thickness of 12 mm and a weight of approximately 11.5 kg/m². The patient table produced in this way has excellent stiffness and strength and exhibits no negative influences on the image quality of the imaging analysis when used in either magnetic resonance spectroscopy or computer tomography.

While the invention has been described with reference to particular embodiments thereof, it will be understood by those having ordinary skill the art that various changes may be made therein without departing from the scope and spirit of the invention. Further, the present invention is not limited to the embodiments described herein; reference should be had to the appended claims.

1-9. (canceled)

10. A patient table for diagnostic equipment comprising:
at least one self-reinforced plastic.

11. The patient table as recited in claim 10, wherein the at least one self-reinforced plastic includes at least one polyolefin.

12. The patient table as recited in claim 11, wherein the polyolefin is a high-density polyethylene (HDPE).

13. The patient table as recited in claim 12, wherein the high-density polyethylene includes highly oriented polyethylene ribbons.

14. A method for producing a patient table including at least one self-reinforced plastic comprising:
providing at least one type-specific plastic;
providing ribbon-shaped, highly stretched monofilaments; and
joining the ribbon-shaped, highly stretched monofilaments with the at least one type-specific plastic.

15. The method as recited in claim 14 wherein:
the monofilaments are stacked one on top of another in layers on a mold so as to form a layer assembly;
the layer assembly is packed with the mold into a foil bag;
the foil bag is then evacuated to a pressure of up to 2 mbar;
the foil bag is then inserted into an autoclave and thermoformed for 2 hours at an autoclave pressure of 9.5 bar and a temperature of 130° C.; and
the foil bag is then cooled and removed from the mold.

16. The method as recited in claim 15, wherein the thermoforming is performed in a closed die heated to 130° C. in a press at a pressing pressure of approximately 10 bar.

17. The method as recited in claim 15, wherein the mold is a female mold.

18. The method as recited in claim 15, wherein the stacking includes stacking the monofilaments one on top of another alternatively in alternately in a 0° direction, a 90° direction and a ±45° direction.
